(19) United States
(12) Patent Application Publication
(10) Pub. No.: US 2019/0167151 A1
(43) Pub. Date: Jun. 6, 2019

(54) SYSTEM AND METHOD FOR TRACKING PATIENT MOVEMENT DURING GUIDED MEDICAL PROCEDURE

(71) Applicant: Acclarent, Inc., Irvine, CA (US)

(72) Inventors: Jetmir Palushi, Irvine, CA (US); Don Q. Ngo-Chu, Irvine, CA (US); Itzhak Fang, Irvine, CA (US)

(21) Appl. No.: 15/831,618

(22) Filed: Dec. 5, 2017

(51) Int. Cl. A61B 5/06; A61B 34/20

(52) U.S. Cl. CPC A61B 5/066; A61B 90/361; A61B 34/20; A61B 5/062

(57) ABSTRACT

A medical instrument includes an instrument position sensor operable to generate an instrument position signal. A first head position sensor is configured to be secured in a first fixed location on or in a patient's head and generate a first head position signal. Field generating elements are configured to generate an electromagnetic field around the patient's head. A processor processes the instrument position signal to determine positioning of the patient's instrument within the electromagnetic field. The processor also processes the first head position signal to determine positioning of the patient's head within the electromagnetic field. The processor determines the position of the instrument within the patient's head based on the determined position of the patient's instrument within the electromagnetic field and based further on the determined position of the patient's head within the electromagnetic field.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc. of Irvine, Calif.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the InstaTrak ENT™ systems available from GE Medical Systems, Salt Lake City, Utah. Other examples of electromagnetic image guidance systems that may be modified for use in accordance with the present disclosure include but are not limited to the CARTO® 3 System by Biosense-Webster, Inc., of Diamond Bar, Calif.; systems available from Surgical Navigation Technologies, Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2-dimensional, line-of-sight view. The use of image guidance systems provides a real time, 3-dimensional view of all of the anatomy surrounding the operative field, not just that which is actually visible in the spatially limited, 2-dimensional, direct line-of-sight endoscopic view. As a result, image guidance systems may be particularly useful during performance of FESS, balloon sinuplasty, and/or other ENT procedures where a section and/or irrigation source may be desirable, especially in cases where normal anatomical landmarks are not present or are difficult to visualize endoscopically.

While several systems and methods have been made and used in ENT procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument assembly, with a guidewire in a proximal position, and with a dilation catheter in a proximal position;

FIG. 1B depicts a perspective view of the dilation instrument assembly of FIG. 1A, with the guidewire in a distal position, and with the dilation catheter in the proximal position;

FIG. 1C depicts a perspective view of the dilation instrument assembly of FIG. 1A, with the guidewire in a distal position, with the dilation catheter in a distal position, and with a dilator of the dilation catheter in a non-dilated state;

FIG. 1D depicts a perspective view of the dilation instrument assembly of FIG. 1A, with the guidewire in a distal position, with the dilation catheter in the distal position, and with a dilator of the dilation catheter in a dilated state;

FIG. 2 depicts a schematic view of an exemplary sinus surgery navigation system;

FIG. 3 depicts a perspective view of the head of a patient, with components of the navigation system of FIG. 2;

FIG. 4 depicts a perspective view of an exemplary medical procedure chair, with an exemplary navigation component support assembly secured to the chair;

FIG. 5 depicts a perspective view of the navigation component support assembly of FIG. 4;

FIG. 6 depicts the medical procedure chair of FIG. 4, with a representation of a patient seated in the chair, and with the patient wearing an exemplary patient tracker system;

FIG. 7 depicts a front elevational view and a side elevational view of earbuds of the patient tracker system of FIG. 6, with each earbud including a multiturn position sensor assembled therein;

FIG. 8 depicts a rear elevational view of the patient tracker system of FIG. 6 assembled onto a representation of a patient's head;

FIG. 9 depicts a side elevational view of the patient tracker system of FIG. 6 assembled onto a representation of a patient's head, with the patient tracker system having a wired connection to a receiver;

FIG. 10 depicts a flow diagram illustrating an algorithm utilized by the patient tracker system of FIG. 6 to determine patient movements and unintentional earbuds movement from the patient's head; and

FIG. 11 depicts a rear elevational view of an exemplary alternative patient tracker system assembled onto a representation of a patient's head, the patient tracker system having a wireless connection to a receiver.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Overview of Exemplary Dilation Catheter System

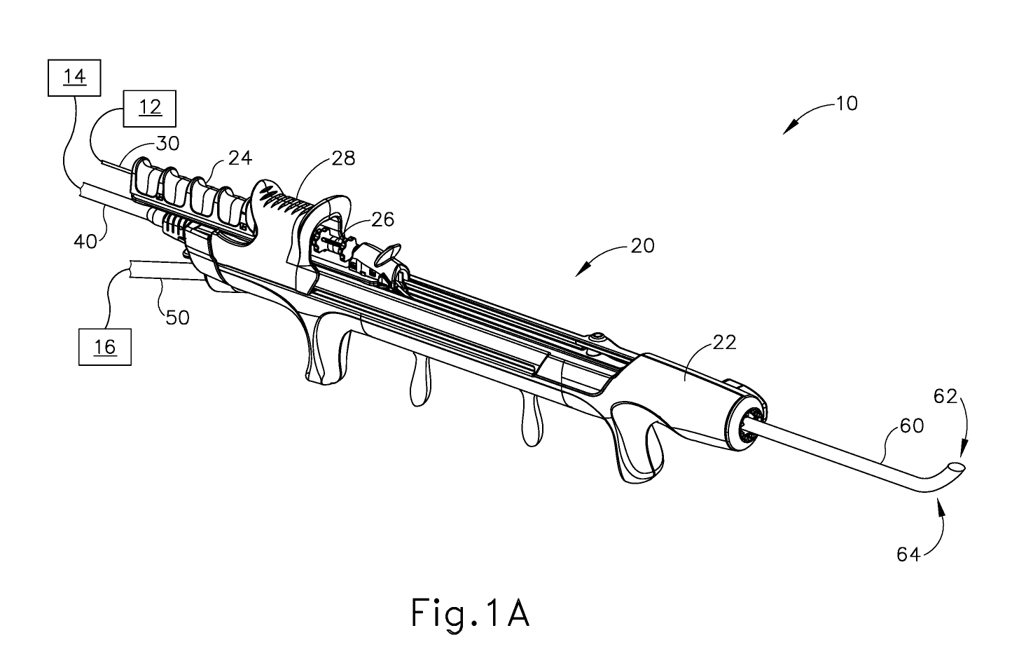

FIGS. 1A-1D shows an exemplary dilation instrument assembly (10) that may be used to dilate the ostium of a paranasal sinus; to dilate some other passageway associated with drainage of a paranasal sinus; to dilate a Eustachian tube; or to dilate some other anatomical passageway (e.g., within the ear, nose, or throat, etc.). Dilation instrument assembly (10) of this example comprises a guidewire power source (12), an inflation source (14), an irrigation fluid source (16), and a dilation instrument (20). In some versions, guidewire power source (12) comprises a source of light. In some other versions, guidewire power source (12) is part of an IGS system as described below. In the present example, inflation source (14) comprises a source of saline. However, it should be understood that any other suitable source of fluid (liquid or otherwise) may be used. Also in the present example, irrigation fluid source (16) comprises a source of saline. Again, though, any other suitable source of fluid may be used. It should also be understood that flush fluid source (16) may be omitted in some versions.

Dilation instrument (20) of the present example comprise a handle body (22) with a guidewire slider (24), a guidewire spinner (26), and a dilation catheter slider (28). Handle body (22) is sized and configured to be gripped by a single hand of a human operator. Sliders (24, 28) and spinner (26) are also positioned and configured to be manipulated by the same hand that grasps handle body (22). It should therefore be understood that dilation instrument (20) may be fully operated by a single hand of a human operator.

### A. Exemplary Guide Catheter

A guide catheter (60) extends distally from handle body (22). Guide catheter (60) includes an open distal end (62) and a bend (64) formed proximal to open distal end (62). In the present example, dilation instrument (20) is configured to removably receive several different kinds of guide catheters (60), each guide catheter (60) having a different angle formed by bend (64). These different angles may facilitate access to different anatomical structures. Various examples of angles and associated anatomical structures are described in one or more of the references cited herein; while further examples will be apparent to those of ordinary skill in the art in view of the teachings herein. Guide catheter (60) of the present example is formed of a rigid material (e.g., rigid metal and/or rigid plastic, etc.), such that guide catheter (60) maintains a consistent configuration of bend (64) during use of dilation instrument (20). In some versions, dilation instrument (20), is further configured to enable rotation of guide catheter (60), relative to handle body (22), about the longitudinal axis of the straight proximal portion of guide catheter (60), thereby further promoting access to various anatomical structures.

### B. Exemplary Guidewire

Dilation instrument (30) further comprises a guidewire (30), which is coaxially disposed in guide catheter (60). Guidewire slider (24) is secured to guidewire (30) such that translation of guidewire slider (24) relative to handle body (22) provides corresponding translation of guidewire (30) relative to handle body (22). In particular, translation of guidewire slider (24) from a proximal position (FIG. 1A) to a distal position (FIG. 1B) causes corresponding translation of guidewire (30) from a proximal position (FIG. 1A) to a distal position (FIG. 1B). When guidewire (30) is in a distal position, a distal portion of guidewire (30) protrudes distally from open distal end (62) of guide catheter (60). Guidewire spinner (26) is operable to rotate guidewire (30) about the longitudinal axis of guidewire (30). Guidewire spinner (26) is coupled with guidewire slider (24) such that guidewire spinner (26) translates longitudinally with guidewire slider (24).

In some versions, guidewire (30) includes a preformed bend formed just proximal to the distal end (32) of guidewire (30). In such versions, the preformed bend and the rotatability provided via guidewire spinner (26) may facilitate alignment and insertion of distal end (32) into a sinus ostium, Eustachian tube, or other passageway to be dilated. Also in some versions, guidewire (30) includes at least one optical fiber extending to a lens or other optically transmissive feature in distal end (32). This optical fiber may be in optical communication with guidewire power source (12), such that light may be communicated from guidewire power source (12) to distal end (32). In such versions, guidewire (30) may provide transillumination through a patient's skin in order to provide visual feedback to the operator indicating that distal end (32) has reached a targeted anatomical structure.

By way of example only, guidewire (30) may be configured in accordance with at least some of the teachings of U.S. Pat. No. 9,155,492, the disclosure of which is incorporated by reference herein. In some versions, guidewire (30) is configured similar to the Relieva Luma Sentry™ Sinus Illumination System by Acclarent, Inc. of Irvine, Calif. In addition to, or as an alternative to, including one or more optical fibers, guidewire (30) may include a sensor and at least one wire that enables guidewire (30) to provide compatibility with an IGS system as described in greater detail below. Other features and operabilities that may be incorporated into guidewire (30) will be apparent to those of ordinary skill in the art in view of the teachings herein.

### C. Exemplary Dilation Catheter

Dilation instrument (30) further comprises a dilation catheter (40), which is coaxially disposed in guide catheter (60). Dilation catheter slider (28) is secured to dilation catheter (40) such that translation of dilation catheter slider (28) relative to handle body (22) provides corresponding translation of dilation catheter (40) relative to handle body (22). In particular, translation of dilation catheter slider (28) from a proximal position (FIG. 1B) to a distal position (FIG. 1C) causes corresponding translation of dilation catheter (40) from a proximal position (FIG. 1B) to a distal position (FIG. 1C). When dilation catheter (40) is in a distal position, a distal portion of dilation catheter (40) protrudes distally from open distal end (62) of guide catheter (60). As can also be seen in FIG. 1C, a distal portion of guidewire (30) protrudes distally from the open distal end of dilation catheter (40) when guidewire (30) and dilation catheter are both in distal positions.

Dilation catheter (40) of the present example comprises a non-extensible balloon (44) located just proximal to open distal end (42) of dilation catheter (40). Balloon (44) is in fluid communication with inflation source (14). Inflation source (14) is configured to communicate fluid (e.g., saline, etc.) to and from balloon (44) to thereby transition balloon (44) between a non-inflated state and an inflated state. FIG. 1C shows balloon (44) in a non-inflated state. FIG. 1D shows balloon (44) in an inflated state. In some versions, inflation source (14) comprises a manually actuated source of pressurized fluid. In some such versions, the manually actuated source of pressurized fluid is configured and operable in accordance with at least some of the teachings of U.S. Pub. No. 2014/0074141, entitled “Inflator for Dilation of Anatomical Passageway,” published Mar. 13, 2014, the disclosure of which is incorporated by reference herein. Other suitable configurations that may be used to provide a source of pressurized fluid will be apparent to those of ordinary skill in the art in view of the teachings herein.

While not shown, it should be understood that dilation catheter (40) may include at least two separate lumens that are in fluid isolation relative to each other. One lumen may provide a path for fluid communication between balloon (44) and inflation source (14). The other lumen may provide a path to slidably receive guidewire (30).

While dilation catheter (40) of the present example is configured to transition between a non-dilated state and a dilated state based on the communication of fluid to and from balloon (44), it should be understood that dilation catheter (40) may include various other kinds of structures to serve as a dilator. By way of example only, balloon (44) may be replaced with a mechanical dilator in some other versions. Dilation catheter (40) may be constructed and operable in accordance with any of the various references cited herein. In some versions, dilator catheter (40) is configured and operable similar to the Relieva Ultirra® Sinus Balloon Catheter by Acclarent, Inc. of Irvine, Calif. In some other versions, dilator catheter (40) is configured and operable similar to the Relieva Solo Pro™ Sinus Balloon Catheter by Acclarent, Inc. of Irvine, Calif. Other suitable variations of dilation catheter (40) will be apparent to those of ordinary skill in the art in view of the teachings herein.

### D. Exemplary Irrigation Features

In some instances, it may be desirable to irrigate an anatomical site. For instance, it may be desirable to irrigate a paranasal sinus and nasal cavity after dilation catheter (40) has been used to dilate an ostium or other drainage passageway associated with the paranasal sinus. Such irrigation may be performed to flush out blood, etc. that may be present after the dilation procedure. In some such cases, guide catheter (60) may be allowed to remain in the patient while guidewire (30) and dilation catheter (40) are removed. A dedicated irrigation catheter (not shown) may then be inserted into guide catheter (60) and coupled with irrigation fluid source (16) via tube (50), to enable irrigation of the anatomical site in the patient. An example of an irrigation catheter that may be fed through guide catheter (60) to reach the irrigation site after removal of dilation catheter (60) is the Relieva Vortex® Sinus Irrigation Catheter by Acclarent, Inc. of Irvine, Calif. Another example of an irrigation catheter that may be fed through guide catheter (60) to reach the irrigation site after removal of dilation catheter (40) is the Relieva Ultirra® Sinus Irrigation Catheter by Acclarent, Inc. of Irvine, Calif.

In some other versions, dilation catheter (40) includes an additional irrigation lumen and an associated set of irrigation ports near distal end (42), such that dilation catheter (40) may be coupled with irrigation fluid source (16) via tube (50). Thus, a separate, dedicated irrigation catheter is not necessarily required in order to provide irrigation.

By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. Of course, irrigation may be provided in the absence of a dilation procedure; and a dilation procedure may be completed without also including irrigation. It should therefore be understood that dilation fluid source (16) and tube (50) are merely optional.

### E. Exemplary Variations

In the present example, guidewire (30) is coaxially disposed within dilation catheter (40), which is coaxially disposed within guide catheter (60). In some other versions, guide catheter (60) is omitted from dilation instrument (20). In some such versions, a malleable guide member is used to guide guidewire (30) and dilation catheter (40). In some such versions, guidewire (30) is omitted and dilation catheter (40) is slidably disposed about the exterior of the internal malleable guide member. In some other versions, guidewire (30) is slidably disposed about the exterior of the internal malleable guide member; and dilation catheter (40) is slidably disposed about the exterior of guidewire (30). In still other versions, guidewire (30) is slidably disposed within the interior of the malleable guide member; and dilation catheter (40) is slidably disposed about the exterior of the malleable guide member.

By way of example only, versions of dilation instrument (20) that include a malleable guide member may be constructed and operable in accordance with at least some of the teachings of U.S. Pub. No. 2016/0310714, entitled “Balloon Dilation System with Malleable Internal Guide,” published Oct. 27, 2016, the disclosure of which is incorporated by reference herein. As another merely illustrative example, versions of dilation instrument (20) that include a malleable guide member may be constructed and operable in accordance with at least some of the teachings of U.S. patent application Ser. No. 14/928,260, entitled “Apparatus for Bending Malleable Guide of Surgical Instrument,” filed Oct. 30, 2015, the disclosure of which is incorporated by reference herein; and/or U.S. Pub. No. 2012/0071857, entitled “Methods and Apparatus for Treating Disorders of the Sinuses,” published Mar. 22, 2012, the disclosure of which is incorporated by reference herein.

It should be understood that the variations of dilation instrument (20) described below in the context of an IGS system may be incorporated into versions of dilation instrument (20) having a malleable guide just like the variations of dilation instrument (20) described below in the context of an IGS system may be incorporated into versions of dilation instrument (20) having a rigid guide catheter (60).

Various examples below describe the use of an IGS system to provide navigation of instruments within a patient. In particular, various examples below describe how dilation instrument assembly (10) may be modified to incorporate IGS system features. However, it should also be understood that dilation instrument assembly (10) may be used in conjunction with conventional image guidance instruments, in addition to being used with IGS system components. For instance, dilation instrument assembly (10) may be used in conjunction with an endoscope, at least to provide initial positioning of guide catheter (60) in a patient. By way of example only, such an endoscope may be configured in accordance with at least some of the teachings of U.S. Pub. No. 2010/0030031, the disclosure of which is incorporated by reference herein. Other suitable kinds of endoscopes that may be used with the various versions of dilation instrument assembly (10) described herein will be apparent to those of ordinary skill in the art.

Other exemplary dilation catheter systems that may be used include the systems described in U.S. Pat. Nos. 8,777,926 and 9,095,646, the disclosures of which are incorporated by reference herein; and the Relieva Ultirra® Sinus Balloon Catheter system by Acclarent, Inc. of Irvine, Calif.

### II. Exemplary Image Guided Surgery Navigation System

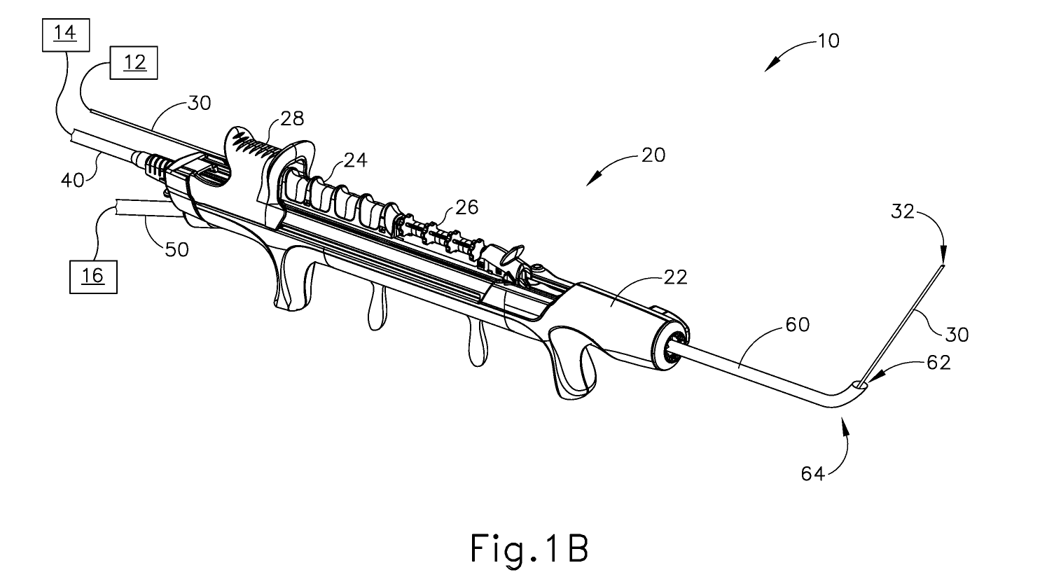

FIG. 2 shows an exemplary IGS navigation system (100) whereby an ENT procedure may be performed using IGS. In some instances, IGS navigation system (100) is used during a procedure where dilation instrument assembly (10) that may be used to dilate the ostium of a paranasal sinus; or to dilate some other anatomical passageway (e.g., within the ear, nose, or throat, etc.). However, it should be understood that IGS navigation system (100) may be readily used in various other kinds of procedures.

In addition to or in lieu of having the components and operability described herein IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

Similarly, in addition to or in lieu of having the components and operability described herein, IGS navigation system (100) may be constructed and operable in accordance with at least some of the teachings of U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. No. 9,198,736, entitled “Adapter for Attaching Electromagnetic Image Guidance Components to a Medical Device,” issued Dec. 1, 2015, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. No. 9,167,961, entitled “Methods and Apparatus for Treating Disorders of the Ear Nose and Throat,” issued Oct. 27, 2015, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

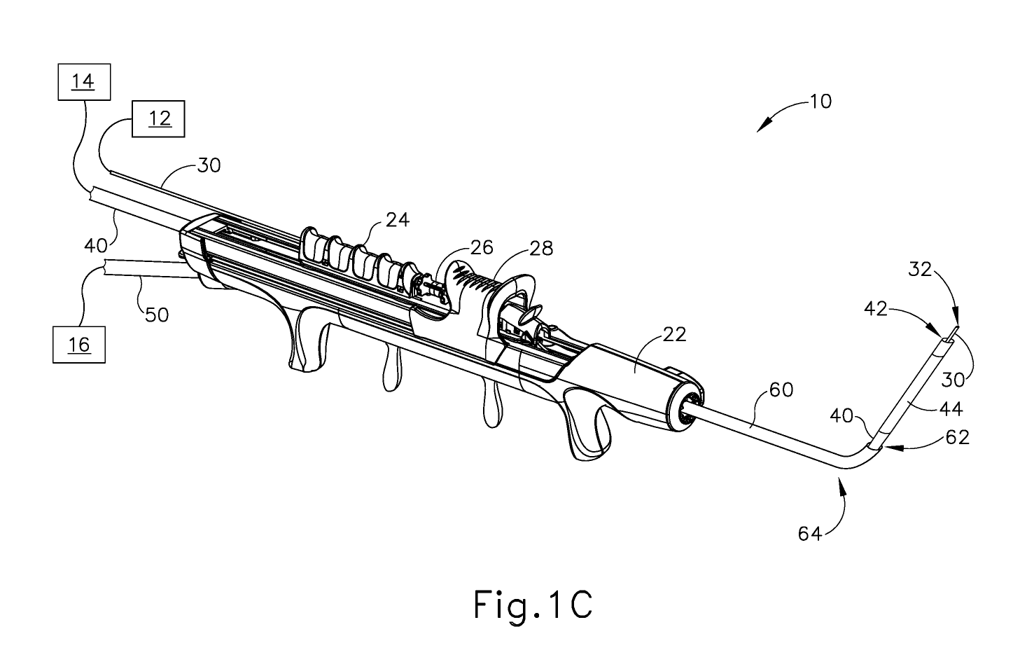

IGS navigation system (100) of the present example comprises a set of magnetic field generators (122). Before a surgical procedure begins, field generators (122) are fixed to the head of the patient. As best seen in FIG. 3, field generators (122) are incorporated into a frame (120), which is clamped to the head of the patient. While field generators (122) are secured to the head of the patient in this example, it should be understood that field generators (122) may instead be positioned at various other suitable locations and on various other suitable structures as will be described in greater detail below. By way of example only, field generators (122) may be mounted on an independent structure that is fixed to a table or chair on which the patient is positioned, on a floor-mounted stand that has been locked in position relative to the head of the patient, and/or at any other suitable location(s) and/or on any other suitable structure(s).

Field generators (122) are operable to generate an electromagnetic field around the head of the patient. In particular, field generators (122) are operated so as to transmit alternating magnetic fields of different frequencies into a region in proximity to frame (120). Field generators (122) thereby enable tracking of the position of a navigation guidewire (130) that is inserted into a nasal sinus of the patient and in other locations within the patient's head. Various suitable components that may be used to form and drive field generators (122) will be apparent to those of ordinary skill in the art in view of the teachings herein.

Navigation guidewire (130) may be used as a substitute for guidewire (30) described above, and may include a sensor (not shown) that is responsive to movement within the fields generated by field generators (122). In particular, signals generated by the sensor of navigation guidewire (130) may be processed by processor (110) to determine the three-dimensional location of navigation guidewire (130) within the patient. Various suitable forms that the sensor may take will be apparent to those of ordinary skill in the art in view of the teachings herein, particularly in view of several of the references that are cited herein in the context of IGS navigation system (100). It should be understood that, when used as a substitute for guidewire (30) in dilation instrument assembly (10), navigation guidewire (130) may facilitate navigation of instrumentation of dilation instrument assembly (10) within the patient during performance of a procedure to dilate the ostium of a paranasal sinus; or to dilate some other anatomical passageway (e.g., within the ear, nose, or throat, etc.). It should also be understood that other components of dilation instrument assembly (10) may incorporate a sensor like the sensor of navigation guidewire (130), including but not limited to the exemplary alternative dilation catheter (200) described below.

IGS navigation system (100) of the present example further comprises a processor (110), which controls field generators (122) and other elements of IGS navigation system (100). Processor (110) comprises a processing unit communicating with one or more memories. Processor (110) of the present example is mounted in a console (116), which comprises operating controls (112) that include a keypad and/or a pointing device such as a mouse or trackball. A physician uses operating controls (112) to interact with processor (110) while performing the surgical procedure.

Console (116) also connects to other elements of system (100). For instance, as shown in FIG. 2 a coupling unit (132) is secured to the proximal end of navigation guidewire (130). Coupling unit (132) of this example is configured to provide wireless communication of data and other signals between console (116) and navigation guidewire (130). In some versions, coupling unit (132) simply communicates data or other signals from navigation guidewire (130) to console (116) uni-directionally, without also communicating data or other signals from console (116). In some other versions, coupling unit (132) provides bidirectional communication of data or other signals between navigation guidewire (130) to console (116). While coupling unit (132) of the present example couples with console (116) wirelessly, some other versions may provide wired coupling between coupling unit (132) and console (116). Various other suitable features and functionality that may be incorporated into coupling unit (132) will be apparent to those of ordinary skill in the art in view of the teachings herein.

Processor (110) uses software stored in a memory of processor (110) to calibrate and operate system (100). Such operation includes driving field generators (122), processing data from navigational guidewire (130), processing data from operating controls (112), and driving display screen (114). The software may be downloaded to processor (110) in electronic form, over a network, for example, or it may, alternatively or additionally, be provided and/or stored on non-transitory tangible media, such as magnetic, optical, or electronic memory.

Processor (110) is further operable to provide video in real time via display screen (114), showing the position of the distal end of navigational guidewire (130) in relation to a video camera image of the patient's head, a CT scan image of the patient's head, and/or a computer generated three-dimensional model of the anatomy within and adjacent to the patient's nasal cavity. Display screen (114) may display such images simultaneously and/or superimposed on each other. Moreover, display screen (114) may display such images during the surgical procedure. Such displayed images may also include graphical representations of instruments that are inserted in the patient's head, such as navigational guidewire (130), such that the operator may view the virtual rendering of the instrument at its actual location in real time. Such graphical representations may actually look like the instrument or may be a much simpler representation such as a dot, crosshairs, etc. By way of example only, display screen (114) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (114). The images provided through display screen (114) may help guide the operator in maneuvering and otherwise manipulating instruments within the patient's head.

In the present example, navigational guidewire (130) includes one or more coils at the distal end of navigational guidewire (130). Such a coil serves as a sensor as referred to above. When such a coil is positioned within an electromagnetic field generated by field generators (122), movement of the coil within that magnetic field may generate electrical current in the coil, and this electrical current may be communicated along the electrical conduit(s) in navigational guidewire (130) and further to processor (110) via coupling unit (132). This phenomenon may enable IGS navigation system (100) to determine the location of the distal end of navigational guidewire (130) within a three-dimensional space as will be described in greater detail below. In particular, processor (110) executes an algorithm to calculate location coordinates of the distal end of navigational guidewire (130) from the position related signals of the coil(s) in navigational guidewire (130).

In some instances, navigational guidewire (130) is used to generate a three-dimensional model of the anatomy within and adjacent to the patient's nasal cavity; in addition to being used to provide navigation for dilation catheter system (100) within the patient's nasal cavity. Alternatively, any other suitable device may be used to generate a three-dimensional model of the anatomy within and adjacent to the patient's nasal cavity before navigational guidewire (130) is used to provide navigation for dilation catheter system (100) within the patient's nasal cavity. By way of example only, a model of this anatomy may be generated in accordance with at least some of the teachings of U.S. Pub. No. 2016/0310042, entitled “System and Method to Map Structures of Nasal Cavity,” published Oct. 27, 2016, the disclosure of which is incorporated by reference herein. Still other suitable ways in which a three-dimensional model of the anatomy within and adjacent to the patient's nasal cavity may be generated will be apparent to those of ordinary skill in the art in view of the teachings herein. It should also be understood that, regardless of how or where the three-dimensional model of the anatomy within and adjacent to the patient's nasal cavity is generated, the model may be stored on console (116). Console (116) may thus render images of at least a portion of the model via display screen (114) and further render real-time video images of the position of navigational guidewire (130) in relation to the model via display screen (114).

### III. Exemplary Support Assembly for Navigation System Components

Some medical procedures, including but not limited to medical procedures that are performed in the ear, nose, or throat of a patient (referred to herein as “ENT procedures”), may be performed while the patient is supported by a chair. As shown in FIGS. 2-3, when an ENT procedure is performed with the assistance of an IGS navigation system (100), it may be necessary to position an array of field generators (122) around the patient's head. In the example described above, field generators (122) are mounted to a frame (120), which is mounted to the patient's head. In this arrangement, when the head of the patient moves, frame (120) and field generators (122) move with the head of the patient, such that the electromagnetic field generated by field generators (122) also moves with the head of the patient. Thus, the frame of reference for IGS navigation system (100) will move with the head of the patient, such that patient head movement will not negatively impact the position data indicating the position of navigational guidewire (130) relative to the head of the patient.

In some instances, it may be desirable to instead position field generators (122) on a support structure that is not mounted to the patient's head. For instance, when the patient is seated in a chair, it may be desirable to have the field generators (122) supported by the structure of the chair rather than being supported by the patient's head. However, mounting field generators (122) to a support structure that is secured to a chair may present other issues that may need to be addressed.

Conventional medical procedure chairs, including those designed particularly for use in ENT procedures, may include several metallic components in the headrest of the chair. While such headrests may provide adequate structural support for field generators (122), metallic components in such headrests (and/or elsewhere within the chair) may interfere with the functioning or accuracy of IGS navigation system (100) if the metallic components are too close to field generators (122). It may therefore be desirable to rely on the chair to structurally support field generators (122) while avoiding the risk of metallic features of the chair compromising the functioning or accuracy of IGS navigation system (100). Moreover, it may be desirable to provide a field generator (122) support assembly that may be readily retrofitted to a conventional medical procedure chair, such that a consumer need not purchase an entire new chair in order to obtain the support functionality described above. In versions where the support assembly may be retrofitted to a conventional medical procedure chair, it may be desirable to enable an operator to accomplish such retrofitting without requiring the use of tools such as screwdrivers, etc.

The following examples relate to support assemblies that may be retrofitted to a conventional medical procedure chair, relying on the chair itself (rather than the patient's head) to structurally support IGS navigation system (100) components such as field generators (122), without the risk of any metallic components of the chair interfering with the functioning or accuracy of IGS navigation system (100), and without requiring the use of separate tools in order to complete the retrofitting.

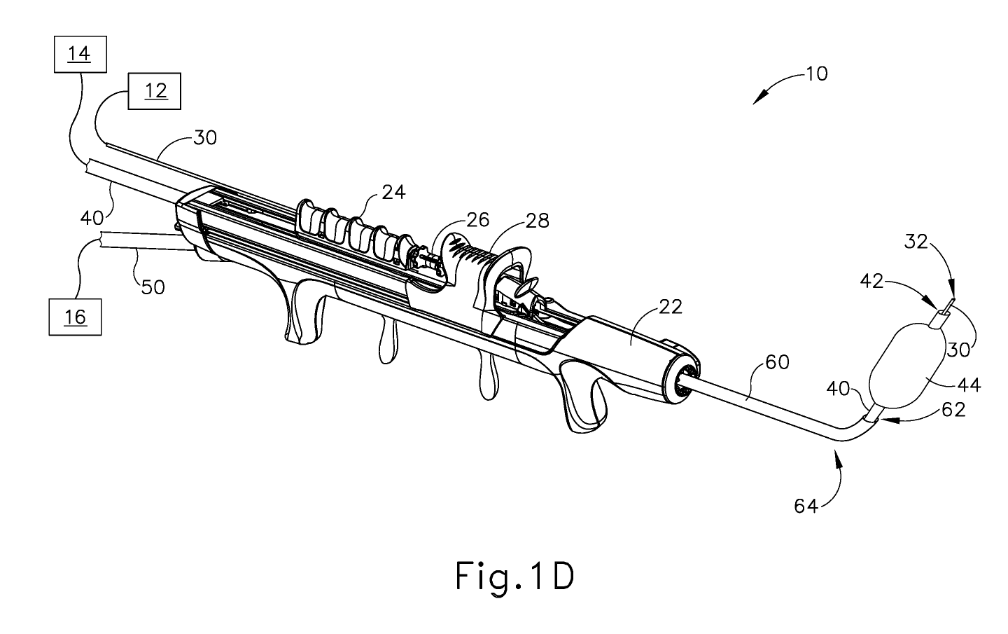

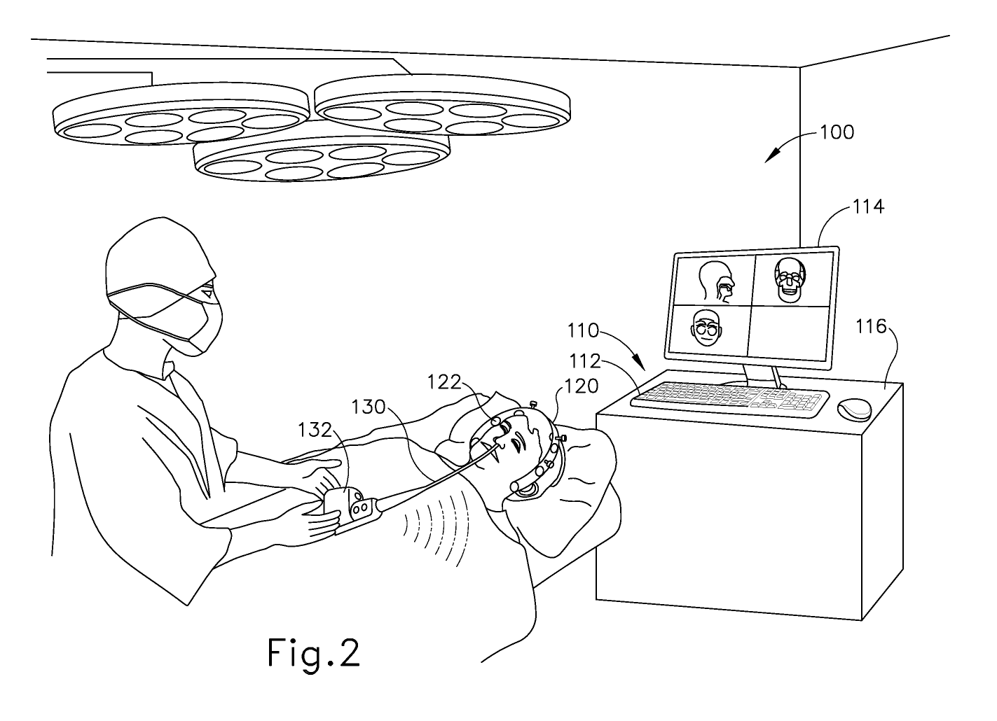

FIG. 4 shows an exemplary ENT procedure chair (200) with a support assembly (300) mounted thereon and supporting a navigation system component (400). Chair (200) includes a base (202), a bottom support (204), a backrest (206), a pair of armrests (208), a headrest (210), and a footrest (212). Chair (200) is configured to seat a patient thereon such that support assembly (300) is positioned adjacent to the patient's head. In particular, headrest (210) is configured to support the head of a patient while the patient is seated on bottom support (204). Support assembly (300) of this example includes a wedge-shaped body (301) that is configured to rest against a front surface (not shown) of backrest (206). A frame (304) extends from and is secured to backrest (206). As best seen in FIG. 5, frame (304) is generally shaped like a horseshoe in this example and includes a plurality of integral field generators (306). Field generators (306) of this example are configured and operable just like field generators (122) described above.

Frame (304) is configured to hold field generators (306) in a generally horseshoe-shaped arrangement about the head of the patient, without frame (304) contacting the patient's head. A cable (not shown) is in communication with field generators (306) and thereby provides a conduit for communication between field generators (306) and processor (110) of IGS navigation system (100). Various features that may be used to secure support assembly (300) to backrest (206) will be apparent to those of ordinary skill in the art in view of the teachings herein.

### IV. Exemplary Patient Tracker System

In the example described above, where field generators (306) are mounted to frame (304), which is thereby assembled to chair (200), rather than on frame (120) which is directly attached to a patient's head, the frame of reference for IGS navigation system (100) (i.e., the electromagnetic field generated by field generators (306)) does not move with the head of the patient. In some instances, a procedure may involve intentional or inadvertent movements by the patient while situated in chair (200), such that the patient's head may shift positions, location, and/or orientation in relation to support assembly (300). When a navigation guidewire (130) (or other instrument having a sensor compatible with IGS navigation system (100)) is disposed in the head of the patient, IGS navigation system (100) may not be able to differentiate between (i) movement of navigation guidewire (130) relative to the head of the patient and (ii) movement of the head of the patient with navigation guidewire (130) (e.g., when navigation guidewire (130) remains stationary relative to the head of the patient yet moves relative to the head of the patient)). Thus, by not securing field generators (306) relative to the head of the patient, IGS navigation system (100) may provide inaccurate position data relative to the head of the patient when the head of the patient moves while navigation guidewire (130) is disposed in the head of the patient. It may therefore be desirable for IGS navigation system (100) include features and functionality to account for movement of the patient's head, to preserve the accuracy of IGS navigation system (100).

For instance, it may beneficial to incorporate navigation system components, such as sensors, onto a patient's head that are configured to communicate with field generators (306) such that movement of a patient's head may be separately tracked and thereby accounted for by IGS navigation system (100). It may further be desirable for a sensor that is dedicated to tracking patient head movement to be unobtrusive, and particularly less obtrusive than frame (120). Less intrusive means of attaching navigation system components onto a patient's head may be beneficial for ease in installation and enhanced comfort for the patient during the procedure.

The following description provides various examples of a navigation system component that is configured to cooperatively communicate with IGS navigation system (100) to improve accuracy in tracking the position of an instrument (e.g., navigation guidewire (130)) that is inserted into the patient's head. In particular, the navigation system component is configured to be responsive to movement of a patient's head in relation to the fields generated by field generators (306), such that the signals generated by a navigational instrument (e.g., navigation guidewire (130)) may be processed by processor (110) through an error correction algorithm, to effectively subtract-out patient head movement, to thereby accurately determine the three-dimensional location of the navigational instrument within the head of the patient.

It should be understood that the navigation system components described below may be readily incorporated into any of the various navigation systems (100) and support assemblies (300) described above and in any of the various medical procedures described in the various references described herein. Other suitable ways in which the below-described navigation system components may be used will be apparent to those of ordinary skill in the art in view of the teachings herein.

### A. Wired Navigation Earbuds

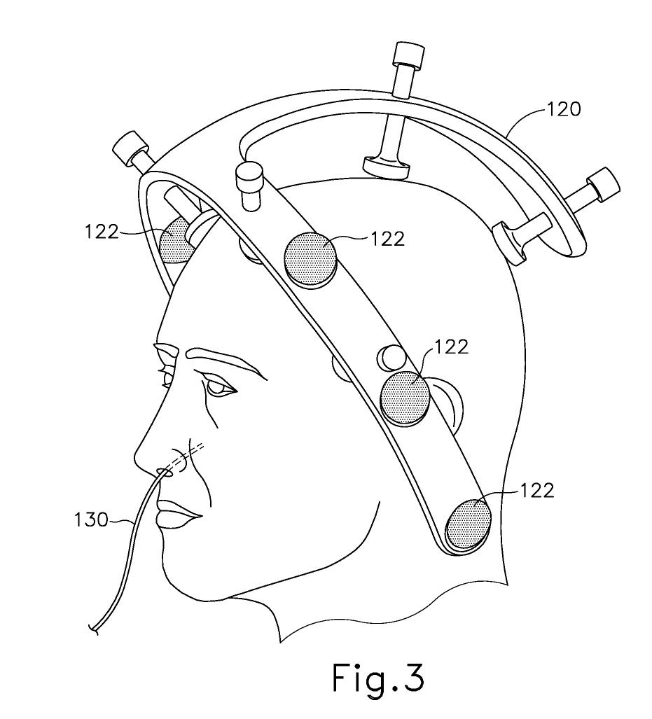

FIG. 6 shows a diagrammatic representation of a patient (P) seated on ENT procedure chair (200) as described above, with a head (H) of patient (P) supported by headrest (342). In particular, head (H) of patient (P) is situated adjacent to support assembly (300) such that the horseshoe-shaped frame (304) extends about head (H) of patient (P) without any field generator (306) contacting head (H) of patient (P). FIG. 6 further shows an exemplary navigation system (500) in electrical communication with support assembly (300). It should be understood that navigation system (500) of the present example may be readily incorporated with support assembly (300) and procedure chair (200) described above. It should also be understood that, in many respects, navigation system (500) may be constructed and operable just like IGS navigation system (100) except for the differences explicitly noted herein. For instance, navigation system (500) comprises a processor (502) that is configured and operable just like processor (110) described above, such that processor (502) comprises a processing unit communicating with one or more memories. As will be described in greater detail below, processor (502) is further operable to communicate with a pair of sensors (516) through a wireless transmitter (530).

Navigation system (500) further comprises a pair of earbuds (510) in communication with wireless transmitter (530) through a respective wire or cable (520). Earbuds (510) are electrical tracking devices that are sized and shaped to securely fit within an ear (E) of patient (P). In this instance, earbuds (510) are ergonomically designed such that either earbud (510) may fit into either ear (E) of patient (P). Alternatively, earbuds (E) may be sized and shaped to correspond to a particular ear (E) (e.g., left ear or right ear) of patient (P) such that each earbud (E) is configured to fit within a designated ear (E) on head (H) of patient (P) during a procedure. Although not shown, earbuds (E) may include a fastening means to enhance the overall attachment of navigation system (500) to patient (P). By way of example only, such fastening means may be in the form of a strap, clip, adhesive, or other various suitable fastening features as will be understood by one of ordinary skill in the art.

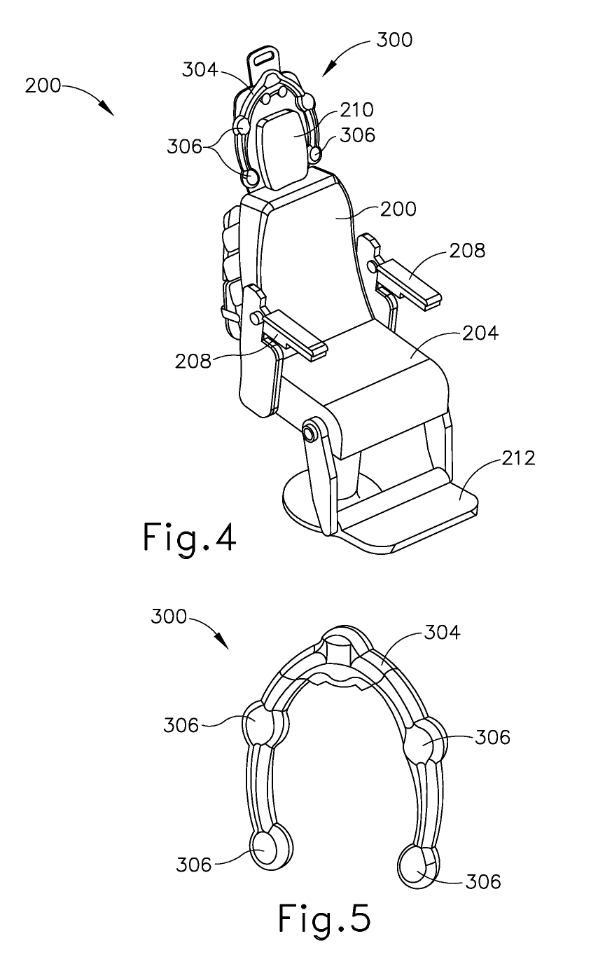

As best seen in FIG. 7, earbuds (510) comprise an occluding member (512), a neck member (514), and a sensor (516). Occluding member (512) is shaped and sized to fit within ear (E) of a patient in an occluding manner. Depending on the size of patient's (P) ear (E), there may be some instances where occluding member (512) does not form an airtight seal when received within ear (E). Neck member (514) extends downwardly from occluding member (512) and is offset from a central axis (not shown) of occluding member (512) such that neck member (514) does not obstruct or inhibit the insertion of occluding member (512) into ear (E) of patient (P). In other words, with neck member (514) extending from occluding member (512) at an alignment offset from the center of occluding member (512), occluding member (512) may be securely received within ear (E) while neck member (514) extends downwardly exterior to ear (E). Wire (520) connects to earbud (510) at a bottom portion of neck member (514) and extends into and through neck member (514) towards occluding member (512) of each respective earbud (510). As will be described in greater detail below, wire (520) is in electrical communication with sensor (516).

Sensor (516) is a multi-turn position sensor that is operable to detect angular movement ranging from 0 degrees to 360 degrees. In other words, sensors (516) comprise position sensing technology that are movement sensitive and configured to provide high-output, high-accuracy, and high-stability tracking while having low-power consumption and low-temperature drifts. By way of example only, sensors (516) may comprise torque and angle sensors (TAS) that are capable of simultaneously providing torque and angle measurements. In some variations, sensors (516) each comprise one or more coils that are configured to generate signals in response to movement of sensors (516) within the electromagnetic field generated by field generators (306). Sensors (516) may thus be configured and operable like the coil(s) integrated into navigation guidewire (130). Other suitable forms that sensors (516) may take will be apparent to those of ordinary skill in the art in view of the teachings herein.

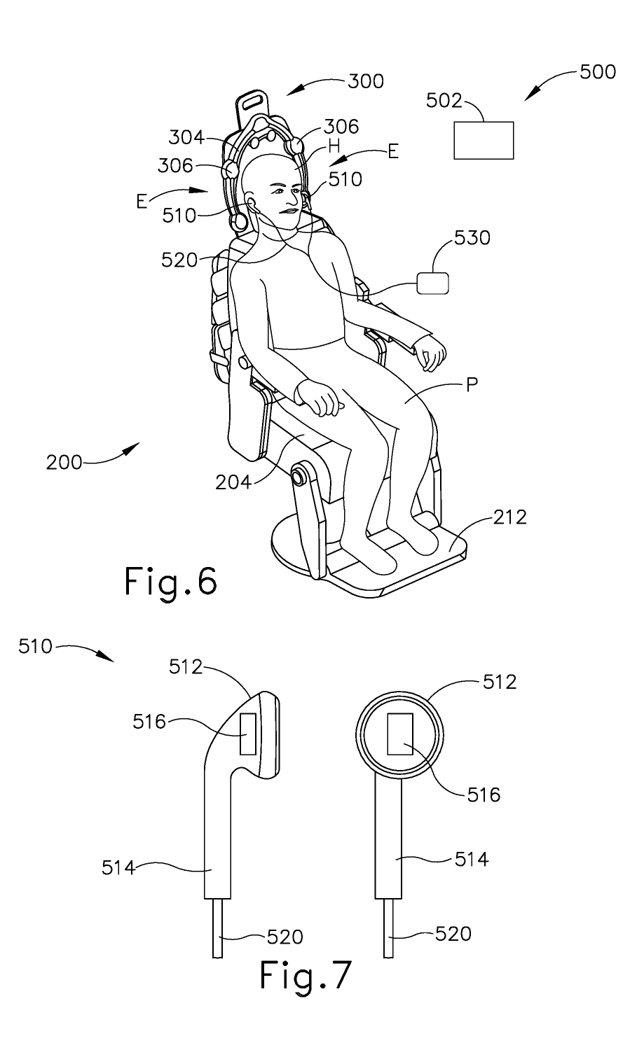

Sensors (516) are located within occluding members (512) of earbuds (510) such that sensors (516) are selectively positioned within ear (E) of patient (P) when occluding members (512) are similarly inserted into ear (E), as seen in FIG. 8. Sensors (512) are in electrical communication with wires (520) such that any torque and angle movements and/or measurements recorded by sensors (516) are communicated through wires (520). The signals from sensors (516) thus reach transmitter (530) via wires (520) in this example.

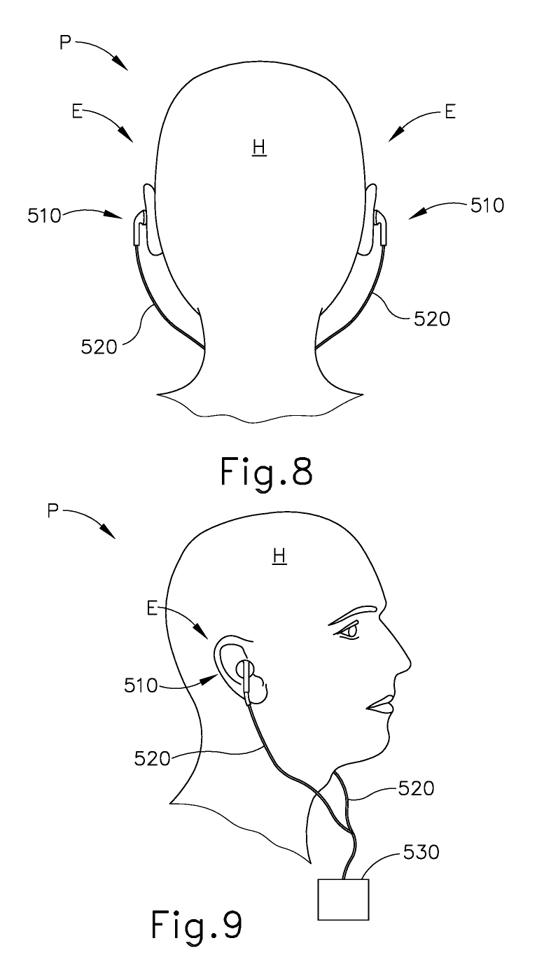

FIG. 9 shows earbud (510) received within ear (E) of patient (P) with wire (520) extending from earbud (510) towards a wireless transmitter (530). Wireless transmitter (530) is operable to wirelessly communicate the data received from sensors (516) of earbuds (510), through wires (520), to processor (502). Wireless transmitter (530) is thus configured to transmit the navigational data recorded by sensors (516) to processor (502), the navigational data being indicative of the current location of earbuds (510) relative to field generators (306) of support assembly (300). In some versions, wireless transmitter (530) may further be configured to supply earbuds (510) with power. In addition, or in the alternative, transmitter (530) may be coupled with processor (502) via one or more wires. As yet another merely illustrative example, earbuds (510) may be directly coupled with processor (502) via wires (520) or wirelessly, such that transmitter (530) may be omitted or integrated into earbuds (510).

Processor (502) is configured to communicate with navigational guidewire (130), earbuds (510), and field generators (306) such that the signals collectively received by processor (502) are processed to determine the three-dimensional location of navigation guidewire (130) within the head (H) of patient (P). In particular, similar to processor (110) described above, processor (502) uses software stored in memory to calibrate and operate navigation system (500) by driving field generators (306), processing data from navigation guidewire (130) and earbuds (510), processing data from operating controls, and driving a display screen similar to display screen (114). Through the display screen, processor (502) is operable to provide video in real time showing the position of the distal end of navigational guidewire (130) in relation to a video camera image of head (H) of patient (P), a CT scan image of head (H) of patient (P) and/or a computer generated three-dimensional model of the anatomy within and adjacent to patient's (P) nasal cavity.

Unlike processor (110), processor (502) of the present example is further configured to identify and filter out movements of head (H) of patient (P) during a procedure when processing and displaying the three-dimensional location of navigation guidewire (130) on the display screen. In other words, in the event one earbud (510) on head (H) of patient (P) moves slightly relative to field generators (306) of support assembly (300), processor (502) is configured to recognize the movement as being inadvertent when the corresponding earbud (510) did not simultaneously move. In this instance, processor (502) disregards the trivial activity of the single earbud (510) when determining the continued location of navigation guidewire (130) in the anatomical passageway.

Processor (502) is further configured to identify and process intentional movements of head (H) of patient (P) during a medical procedure when processing and displaying the three-dimensional location of navigation guidewire (130) on the display screen. In other words, in the event both earbuds (510) on head (H) of patient (P) move simultaneously relative to field generators (306) of support assembly (300), regardless of the degree of movement, processor (502) is configured to recognize the collective movement as being intentional and noteworthy. In this instance, processor (502) factors the movement of both earbuds (510) when determining the continued location of navigation guidewire (130) in the anatomical passageway. Tracking intentional movements of head (H) of patient (P) during a procedure provides improved accuracy and reliability in navigation system (500) to precisely display the location of navigation guidewire (130) to an operator.

In some instances, processor (502) does not attempt to distinguish between unintentional head (H) movement (e.g., only a single earbud (510) moves) and intentional head (H) movement (e.g., both earbuds (510) move). In such instances, processor (502) may factor in any and all detected head (H) movement, regardless of whether one or both earbuds (510) move, in an error correction algorithm to determine the precise position of navigation guidewire within the head (H) of the patient (P).

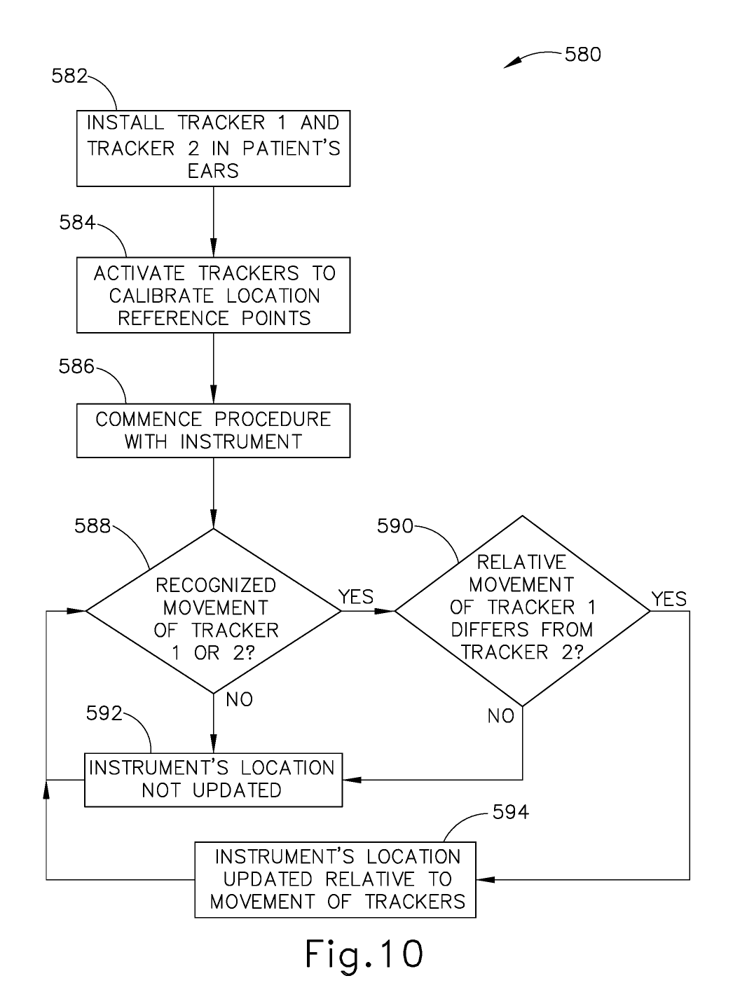

FIG. 10 shows a flow diagram illustrating steps of an exemplary method (580) that may be executed via navigation system (500) with the use of procedure chair (200) and support assembly (300). At step (582), after patient (P) is seated in procedure chair (200) and head (H) of patient (P) is rested against headrest (342) and adjacent to support assembly (300), earbuds (510) (i.e. “tracker 1” and “tracker 2” in FIG. 10) are installed in head (H) of patient (P). In this instance, occluding members (512) are received within respective ears (E) of patient (P). Alternatively, earbuds (510) may be installed in head (H) of patient (P) before patient (P) is seated in chair (200) or at any other suitable time prior to or during the procedure. Earbuds (510) are electrically activated at step (584) such that sensors (516) of each earbud (510) become operational and initiate navigational calibration relative to the other earbud (510) and field generators (306) of support assembly (300).

With sensors (516) sufficiently powered and calibrated, an operator may commence the medical procedure and insert various navigational instruments, such as navigation guidewire (130) described above, into head (H) of patient (P), as seen in step (586). As described in detail above, navigation guidewire (130) includes a sensor that is responsive to movement within the fields generated by field generators (306). In this instance, signals generated by the sensor of navigation guidewire (130) are processed by processor (502) to determine the three-dimensional location of navigation guidewire (130) within the field generated by field generators (306). So long as the head (H) of the patient (P) remains substantially motionless within the field generated by field generators (306), the location of navigation guidewire (130) within the field generated by field generators (306) will be accurately indicative of the location of navigation guidewire (130) within the head (H) of the patient (P). Sensors (516) of earbuds (510) remain in constant communication with processor (502) as field generators (306) simultaneously communicate with processor (502).

At step (588), processor (502) continuously monitors the location of sensors (516) of earbuds (510) through wireless transmitter (530) to determine if either earbud (510) moves during the medical procedure. When processor (502) detects the movement of one earbud (510) relative to field generators (306) of support assembly (300), processor (502) then evaluates whether the other earbud (510) similarly moved with respect to its initial position in the field, as seen at step (590). If the other earbud (510) did not move simultaneously with the initial earbud (510) that triggered step (588), then processor (502) identifies the movement as being inadvertent and trivial such that the processed location of navigation guidewire (130), as displayed on the display screen, is not updated as seen at step (592) based on movement of the head (H). However, if the sensor (516) of the other earbud (510) transmits data through wireless transmitter (530) to processor (502) indicating simultaneous movement with the earbud (510) that initially triggered step (588), then processor (502) identifies the movement as being intentional such that the location of navigation guidewire (130) is reprocessed and updated on the display screen based on movement of the head (H), as seen at step (594).

In this instance, after reprocessing the accurate location of navigation guidewire (130), processor (502) continues to monitor the location of sensors (516) of earbuds (510) relative to field generators (306) to determine if either earbud (510) moves again during the medical procedure. Processor (502) continues this exemplary method (580) for as long as navigation system (500) is in operation. Ultimately, navigation system (500) constantly monitors movement of head (H) of patient (P) as navigation guidewire (130) is utilized within the anatomical passageways of head (H), to thereby accurately depict the position of navigation guidewire (130) within the head (H) throughout the duration of the procedure. Earbuds (510) serve to give processor (502) with multiple reference points of head (H) of patient (P) to thereby more accurately track the movement of head (H) during the procedure while incorporating fault tolerant software to disregard inadvertent movements of head (H).

### B. Wireless Navigation Earbuds

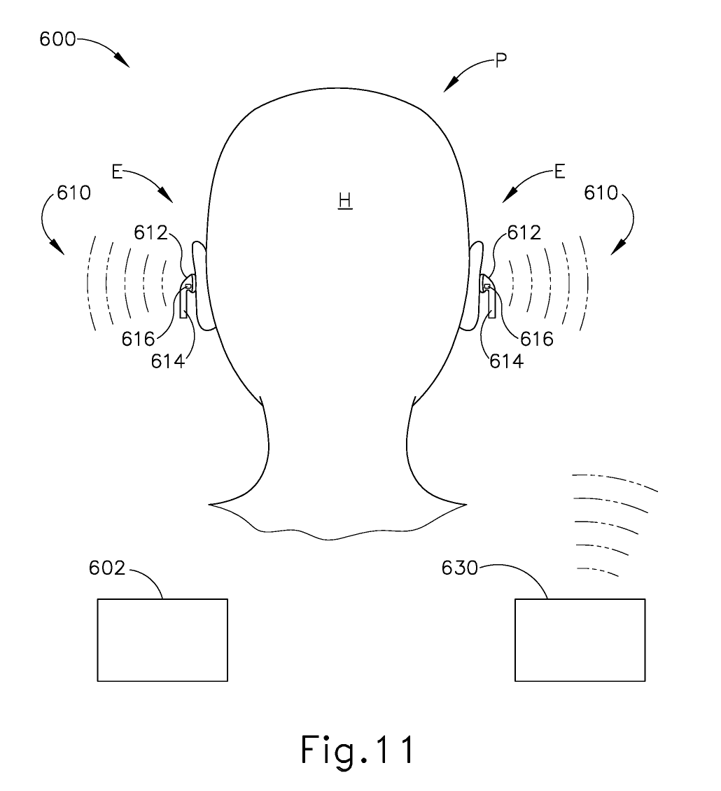

FIG. 11 shows another exemplary navigation system (600). Except as otherwise provided below, it should be understood that navigation system (600) is operable and configured similar to navigation system (500) described above. It should be further understood that navigation system (600) of the present example may be readily incorporated with support assembly (300) and procedure chair (200) described above. In many respects, navigation system (600) functions substantially similar to navigation system (500). For instance, navigation system (600) comprises a processor (602), a pair of earbuds (610), and a wireless transmitter (630). It should be understood that processor (602), earbuds (610), and wireless transmitter (630) are configured and operable just like processor (502), earbuds (510), and wireless transmitter (530) described above, respectively, except for the differences explicitly described herein. Similar to earbuds (510), each earbud (610) comprises an occluding member (612), a neck member (614), and a sensor (616), with sensor (616) integrally positioned within the occluding member (612). Unlike earbuds (510), neck members (614) do not have a wire or cable extending therefrom to connect sensors (616) to wireless transmitter (630). Rather, in the present example, earbuds (610) are configured to communicate with wireless transmitter (630) through a wireless connection such that sensors (616) are operable to transmit data to wireless transmitter (630) without a wire or cable.

In the present example, notwithstanding earbuds (610) being operable to communicate navigational data from sensors (616) to wireless transmitter (630) through a wireless transmission, navigation system (600) is operable to be similarly utilized in method (580) described above, just like navigation system (500), with procedure chair (200) and support assembly (300) as shown in FIG. 10. In this instance, however, wireless transmitter (630) will receive the signals generated by sensors (616) through a wireless connection prior to transmitting the data to processor (602) for processing. In other versions, processor (602) may be configured to communicate directly with earbuds (610) through a wireless transmission such that the inclusion of wireless transmitter (630) is not necessary in navigation system (600). In that instance, the signals generated by sensors (616) are communicated directly to processor (602).

### V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A system comprising: (a) a medical instrument, wherein the medical instrument is configured to be inserted into a patient's head, wherein the medical instrument includes an instrument position sensor operable to generate an instrument position signal; (b) a first head position sensor, wherein the first head position sensor is configured to be secured in a first fixed location on or in a patient's head, wherein the first head position sensor is configured to generate a first head position signal; (c) a plurality of field generating elements, wherein the field generating elements are configured to be positioned around a patient's head such that the field generating elements are configured to generate an electromagnetic field around the patient's head; and (e) a processor, wherein the processor is in communication with the instrument position sensor, the first head position sensor, and the field generating elements, wherein the processor is operable to process the instrument position signal to determine positioning of the patient's instrument within the electromagnetic field, wherein the processor is operable to process the first head position signal to determine positioning of the patient's head within the electromagnetic field, wherein the processor is further configured to determine the position of the instrument within the patient's head based on the determined position of the patient's instrument within the electromagnetic field and based further on the determined position of the patient's head within the electromagnetic field.

### EXAMPLE 2

The system of Example 1, further comprising an earbud configured to fit in a patient's ear, wherein the first head position sensor is located in the earbud.

### EXAMPLE 3

The system of any one or more of Examples 1 through 2, further comprising a transmitter, wherein the transmitter in communication with the first head position sensor and the processor, wherein the transmitter is configured to transmit the first head position signal to the processor.

### EXAMPLE 4

The system of Example 3, wherein the transmitter is in communication with the first head position sensor via one or more wires.

### EXAMPLE 5

The system of Example 3, wherein the transmitter is in communication with the head position sensor wirelessly.

### EXAMPLE 6

The system of Example 3 or Example 4, wherein the transmitter is in communication with the processor via one or more wires.

### EXAMPLE 7

The system of Example 3 or Example 4, wherein the transmitter is in communication with the processor wirelessly.

### EXAMPLE 8

The system of any one or more of Examples 1 through 7, further comprising a second head position sensor, wherein the second head position sensor is configured to be secured in a second fixed location on or in a patient's head, wherein the second head position sensor is configured to generate a second head position signal, wherein the processor is in communication with the second head position sensor, wherein the processor is operable to process the second head position signal to further determine positioning of the patient's head within the electromagnetic field.

### EXAMPLE 9

The system of Example 8, further comprising a first earbud and a second earbud, wherein the first head position sensor is located in the first earbud, wherein the second head position sensor is located in the second earbud.

### EXAMPLE 10

The system of any one or more of Examples 8 through 9, wherein the processor is further configured to determine whether patient head movement is intentional, based on whether movement of the first head position sensor differs from movement of the second head position sensor.

### EXAMPLE 11

The system of Example 10, wherein the processor is configured to effectively disregard the head position signals in response to determining that patient head movement is unintentional, wherein the processor is further configured to determine the position of the instrument within the patient's head based on the determined position of the patient's instrument within the electromagnetic field, based on the first head position signal, and based on the second head position signal.

### EXAMPLE 12

The system of any one or more of Examples 1 through 11, wherein the medical instrument comprises a guidewire.

### EXAMPLE 13

The system of Example 12, wherein the instrument position sensor is located at a distal end of the guidewire.

### EXAMPLE 14

The system of any one or more of Examples 1 through 11, wherein the medical instrument comprises a dilation catheter.

### EXAMPLE 15

The system of any one or more of Examples 1 through 14, wherein the instrument position sensor comprises a coil configured to generate a signal based on movement of the coil within the electromagnetic field.

### EXAMPLE 16

The system of any one or more of Examples 1 through 15, wherein the first head position sensor comprises a coil configured to generate a signal based on movement of the coil within the electromagnetic field.

### EXAMPLE 17

The system of any one or more of Examples 1 through 16, further comprising a chair configured to support a patient, wherein the field generating elements are fixedly secured to the chair.

### EXAMPLE 18

The system of any one or more of Examples 1 through 17, further comprising a curved frame supporting the field generating elements, wherein the curved frame is configured to form an arcuate shape near a patient's head.

### EXAMPLE 19

A method comprising: (a) securing a head position sensor to a patient's head; (b) inserting a medical instrument in the patient's head, wherein the medical instrument includes a medical instrument sensor; (c) activating a navigation system, wherein the activated navigation system: (i) generates an electromagnetic field around the patient's head, (ii) determines the position of the medical instrument within the electromagnetic field based on a signal from the medical instrument sensor, (iii) determines the position of the patient's head within the electromagnetic field based on a signal from the medical instrument sensor, and (iv) determines the position of the medical instrument within the patient's head based on at least: (A) the determined position of the medical instrument within the electromagnetic field, and (B) the determined position of the patient's head within the electromagnetic field.

### EXAMPLE 20

A method, comprising: (a) generating an electromagnetic field around the patient's head; (b) receiving a medical instrument position signal from a medical instrument position sensor indicating a position of the medical instrument in the electromagnetic field; (c) receiving a head position signal from a head position sensor indicating a position of the patient's head in the electromagnetic field, wherein the head position sensor is fixedly secured to the patient's head; and (d) determining the position of the medical instrument in the patient's head based on at least: (i) the received medical instrument position signal, and (ii) the received head position signal.

### EXAMPLE 21

A method comprising: (a) securing a head position sensor to a patient's head; (b) inserting a medical instrument in the patient's head via the patient's ear, nose, or mouth, wherein the medical instrument includes a medical instrument sensor; (c) activating a navigation system, wherein the activated navigation system: (i) generates an electromagnetic field around the patient's head, (ii) determines the position of the medical instrument within the electromagnetic field based on a signal from the medical instrument sensor, (iii) determines the position of the patient's head within the electromagnetic field based on a signal from the medical instrument sensor, and (iv) determines the position of the medical instrument within the patient's head based on at least: (A) the determined position of the medical instrument within the electromagnetic field, and (B) the determined position of the patient's head within the electromagnetic field.

### EXAMPLE 22

The method of Example 21, wherein the medical instrument comprises a guide member and a dilation catheter, wherein the act of inserting the medical instrument in the patient's head comprises inserting the guide member in the patient's head.

### EXAMPLE 23

The method of Example 22, wherein the dilation catheter includes an expandable element, the method further comprising: (a) advancing the dilation catheter relative to the guide member to position the expandable element at a targeted anatomical structure of the patient; and (b) expanding the expandable element against the targeted anatomical structure.

### EXAMPLE 24

The method of Example 23, wherein the targeted anatomical structure is selected from the group consisting of a Eustachian tube or a drainage passageway associated with a paranasal sinus.

### EXAMPLE 25

The method of Example 24, wherein the targeted anatomical structure comprises paranasal sinus ostium.

### EXAMPLE 26

The method of Example 22, wherein the medical instrument sensor is positioned at a distal end of the guide member, wherein the activated navigation system determines the position of the distal end of the guide member within the electromagnetic field based on a signal from the medical instrument sensor.

### EXAMPLE 27

The method of Example 22, wherein the medical instrument sensor is positioned at a distal end of the dilation catheter, wherein the activated navigation system determines the position of the distal end of the dilation catheter within the electromagnetic field based on a signal from the medical instrument sensor.

### EXAMPLE 28

The method of Example 21, wherein the medical instrument comprises a guidewire, wherein the act of inserting the medical instrument in the patient's head comprises inserting the guidewire in the patient's head.

### EXAMPLE 29

The method of Example 28, wherein the medical instrument sensor is positioned at a distal end of the guidewire, wherein the activated navigation system determines the position of the distal end of the guidewire within the electromagnetic field based on a signal from the medical instrument sensor.

### EXAMPLE 30

The method of any one or more of Examples 21 through 29, the method further comprising positioning the patient in a chair, wherein one or more electromagnetic field generators are secured to the chair, wherein the one or more electromagnetic field generators secured to the chair generate the electromagnetic field around the patient's head.

### EXAMPLE 31

The method of any one or more of Examples 21 through 30, wherein the head position sensor is located in one or more earbuds, wherein the act of securing the head position sensor to the patient's head comprises inserting the one or more earbuds in the patient's ear or ears.

### VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A system comprising:
(a) a medical instrument, wherein the medical instrument is configured to be inserted into a patient's head, wherein the medical instrument includes an instrument position sensor operable to generate an instrument position signal;
(b) a first head position sensor, wherein the first head position sensor is configured to be secured in a first fixed location on or in a patient's head, wherein the first head position sensor is configured to generate a first head position signal;
(c) a plurality of field generating elements, wherein the field generating elements are configured to be positioned around a patient's head such that the field generating elements are configured to generate an electromagnetic field around the patient's head; and
(d) a processor, wherein the processor is in communication with the instrument position sensor, the first head position sensor, and the field generating elements,
wherein the processor is operable to process the instrument position signal to determine positioning of the patient's instrument within the electromagnetic field,
wherein the processor is operable to process the first head position signal to determine positioning of the patient's head within the electromagnetic field,
wherein the processor is further configured to determine the position of the instrument within the patient's head based on the determined position of the patient's instrument within the electromagnetic field and based further on the determined position of the patient's head within the electromagnetic field.

2. The system of claim 1, further comprising an earbud configured to fit in a patient's ear, wherein the first head position sensor is located in the earbud.

3. The system of claim 1, further comprising a transmitter, wherein the transmitter in communication with the first head position sensor and the processor, wherein the transmitter is configured to transmit the first head position signal to the processor.

4. The system of claim 3, wherein the transmitter is in communication with the first head position sensor via one or more wires.

5. The system of claim 3, wherein the transmitter is in communication with the head position sensor wirelessly.

6. The system of claim 3, wherein the transmitter is in communication with the processor via one or more wires.

7. The system of claim 3, wherein the transmitter is in communication with the processor wirelessly.

8. The system of claim 1, further comprising a second head position sensor, wherein the second head position sensor is configured to be secured in a second fixed location on or in a patient's head, wherein the second head position sensor is configured to generate a second head position signal, wherein the processor is in communication with the second head position sensor, wherein the processor is operable to process the second head position signal to further determine positioning of the patient's head within the electromagnetic field.

9. The system of claim 8, further comprising a first earbud and a second earbud, wherein the first head position sensor is located in the first earbud, wherein the second head position sensor is located in the second earbud.

10. The system of claim 8, wherein the processor is further configured to determine whether patient head movement is intentional, based on whether movement of the first head position sensor differs from movement of the second head position sensor.

11. The system of claim 10, wherein the processor is configured to effectively disregard the head position signals in response to determining that patient head movement is unintentional,
wherein the processor is further configured to determine the position of the instrument within the patient's head based on the determined position of the patient's instrument within the electromagnetic field, based on the first head position signal, and based on the second head position signal.

12. The system of claim 1, wherein the medical instrument comprises a guidewire.

13. The system of claim 12, wherein the instrument position sensor is located at a distal end of the guidewire.

14. The system of claim 1, wherein the medical instrument comprises a dilation catheter.

15. The system of claim 1, wherein the instrument position sensor comprises a coil configured to generate a signal based on movement of the coil within the electromagnetic field.

16. The system of claim 1, wherein the first head position sensor comprises a coil configured to generate a signal based on movement of the coil within the electromagnetic field.

17. The system of claim 1, further comprising a chair configured to support a patient, wherein the field generating elements are fixedly secured to the chair.

18. The system of claim 1, further comprising a curved frame supporting the field generating elements, wherein the curved frame is configured to form an arcuate shape near a patient's head.

19. A method comprising:
(a) securing a head position sensor to a patient's head;
(b) inserting a medical instrument in the patient's head, wherein the medical instrument includes a medical instrument sensor;
(c) activating a navigation system, wherein the activated navigation system:
(i) generates an electromagnetic field around the patient's head,
(ii) determines the position of the medical instrument within the electromagnetic field based on a signal from the medical instrument sensor,
(iii) determines the position of the patient's head within the electromagnetic field based on a signal from the medical instrument sensor, and
(iv) determines the position of the medical instrument within the patient's head based on at least:
(A) the determined position of the medical instrument within the electromagnetic field, and
(B) the determined position of the patient's head within the electromagnetic field.

20. A method, comprising:
(a) generating an electromagnetic field around a patient's head;
(b) receiving a medical instrument position signal from a medical instrument position sensor indicating a position of the medical instrument in the electromagnetic field;
(c) receiving a head position signal from a head position sensor indicating a position of the patient's head in the electromagnetic field, wherein the head position sensor is fixedly secured to the patient's head; and
(d) determining the position of the medical instrument in the patient's head based on at least:
(i) the received medical instrument position signal, and
(ii) the received head position signal.
